(19) United States
(12) Patent Application Publication
(10) Pub. No.: US 2004/0245135 A1
(43) Pub. Date: Dec. 9, 2004

(54) Method and system for collecting used medical devices

(76) Inventor: Ronald Nordquist, Deephaven, MN (US)

Correspondence Address: Sean D. Solberg, DORSEY & WHITNEY LLP, Intellectual Property Department, 50 South Sixth Street, Suite 1500, Minneapolis, MN, 55402-1498, US

(21) Appl. No.: 10/798,241

(22) Filed: Mar. 11, 2004

Related U.S. Application Data
(60) Provisional application No. 60/462,403, filed on Apr. 11, 2003.
(60) Provisional application No. 60/520,710, filed on Nov. 17, 2003.

(51) Int. Cl. A61B 17/06

(52) U.S. Cl. 206/438

(57) ABSTRACT

The subject invention is a method and apparatus for collecting medical devices. The apparatus according to one embodiment is a collection tray having a flexible base having at least one aperture and at least one flexible wall. The method according to one embodiment includes removing a collection container from a sterilized package at a point of use and placing a used medical device in the container.

### CROSS-REFERENCE TO RELATED APPLICATION(S)

This application claims priority from U.S. Provisional Application No. 60/462,403 entitled “Collection Tube for Single Use Medical Devices,” filed on Apr. 11, 2003, and U.S. Provisional Application No. 60/520,710 entitled “Medical Instrument Collection Tray,” filed on Nov. 17, 2003, the contents of both of which are hereby incorporated in their entirety.

### TECHNICAL FIELD

The present invention relates generally to medical devices and in particular the present invention relates to methods and apparatuses for collection of single-use medical devices.

### BACKGROUND OF THE INVENTION

In a sterile environment such as an operating room in a medical facility or hospital, all instruments and personnel must be sterile to avoid the potential for infection in patients. This is especially true in the case of instruments.

Medical instruments range in price and complexity from items costing only a few cents and consisting of no moving parts, to extremely expensive and/or complicated items that may cost upwards of several thousand dollars. Many such devices are primarily designed to be single-use medical devices, that is, they are used once, and then discarded. Some of these single-use medical devices area are quite expensive, with some reaching prices in excess of several thousand dollars.

Devices that are typically single-use devices include by way of example but not by way of limitation, EP catheters, laparoscopy instruments, biopsy forceps, catheters in general, trocars, shaver blades, burrs, bits, and the like. In a typical hospital environment, such single-use medical devices are used and discarded, and new items are used for a next procedure. With the increasing costs of medical equipment, and the continued pressure from the insurance industry and the like to cut health care costs, it would be desirable to provide a lower cost alternative to new single-use medical devices.

To effect such a lower cost alternative, companies have begun to develop protocols and methods for the reprocessing of single-use medical devices. Such reprocessing typically comprises collecting, receiving, cleaning, testing, packaging, sterilization, and reshipping. Current procedures for collecting the used single-use medical devices involve hospital personnel removing devices from sterile areas or waste collection depositories outside of sterile areas when they are no longer being used. This creates a need for extra personnel in the hospital, or for extra work for existing personnel, and creates an inconvenience that typically leads to a lower collection rate for used devices.

Still further, the used devices may have biological material thereon, and the refuse containers in which the devices are placed may also contain other undesirable or contaminating objects. This also negatively affects the collection rate for discarded single-use medical devices.

For the reasons stated above, and for other reasons stated below which will become apparent to those skilled in the art upon reading and understanding the present specification, there is a need in the art for an improved method and apparatus for collection of single-use medical devices that are to be reprocessed.

### BRIEF SUMMARY OF THE INVENTION

The present invention, in one embodiment, is a collection container for used medical devices. The container has a collection body comprising a porous composition, the collection body defining an opening, and a cap configured to be removably coupleable with the collection body at the opening.

In a further embodiment, the present invention is a collection container for used medical devices. The collection container has a collection body comprising a porous composition. The container also has first and second openings, the first opening defined at a first end of the collection body and the second opening defined at a second end of the collection body. The container also has first and second caps configured to be interchangeably removably coupleable with the collection body at the first and second ends.

The present invention, in another embodiment, is a collection container for used medical devices. The container has a collection body comprising first and second open ends and first and second caps configured to be interchangeably removably coupleable with the collection body at the first and second open ends.

In an additional embodiment, the present invention is a collection package for the collection of used medical devices. The package has a collection container having a collection body defining a first opening and a first cap configured to be removably coupleable with the collection body at the first opening. The collection package also has a package configured to removably receive the collection container, the package further configured to maintain sterility of the collection container before removal.

The present invention, in a further embodiment, is a collection container for used medical devices. The collection container has a flexible base defining at least one aperture; and at least one flexible wall coupled to the base, the at least one wall defining an opening in the container.

Additionally, the present invention according to one embodiment is a collection package for the collection of used medical devices. The collection package has a collection container comprising a flexible base defining at least one aperture and at least one flexible wall coupled to the base, the at least one wall defining an opening in the container. The collection package also has a package configured to removably receive the collection container, the packager further configured to maintain sterility of the collection container before removal.

The present invention, in one embodiment, is a system for collecting used medical devices. The system has at least one first collection container and at least one second collection container. The first collection container has a collection body defining a first opening, and a first cap configured to be removably coupleable with the collection body at the first opening. The second collection container has a flexible base defining at least one aperture, and at least one flexible wall coupled to the base, the at least one wall defining an opening in the second collection container.

In another embodiment, the present invention is a method of collecting used medical devices. The method includes removing a collection container from a sterilized package at a point of use, and placing at least one used medical device in the container.

The present invention, in an additional embodiment, is a method of collecting used single-use medical devices. The method includes removing a first collection container from a first sterilized package at a point of use, placing at least one used medical device in the first container, removing a second collection container from a second sterilized package at the point of use, and placing at least one used medical device in the second container. The first container has a collection body defining a first opening, and a first cap configured to be removably coupleable with the collection body at the first opening. The second container has a flexible base defining at least one aperture, and at least one flexible wall coupled to the base, the at least one wall defining an opening in the container.

Additionally, the present invention is a method of cleaning used medical devices. The method includes providing a sterilized package containing a collection container, removing the collection container from the sterilized package, placing at least one used medical device in the collection container, and cleaning the at least one used medical device in the collection container.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is perspective view of a relatively rigid collection container, according to one embodiment of the present invention.

FIG. 2 is a side elevation view of a portion of a relatively rigid collection container, according to one embodiment of the present invention.

FIG. 3 is a perspective view of a relatively rigid collection container, according to an alternative embodiment of the present invention.

FIGS. 4, 5, and 6 depict end elevation views of relatively rigid collection containers, according to alternative embodiments of the present invention.

FIG. 7 shows a front view of a sterilized point-of-use collection tube package, according to one embodiment of the present invention.

FIG. 7A depicts a side view of a sterilized point-of-use collection tube package, according to one embodiment of the present invention.

FIG. 8 is a front view of a decontamination bath, according to one embodiment of the present invention.

FIG. 8A is a flow diagram depicting a method of using a relatively rigid collection container, according to one embodiment of the present invention.

FIG. 9 is an elevation view of a collection tube with devices placed therein, according to one embodiment of the present invention.

FIG. 10 is an elevation view of a collection tube with devices placed therein and an end cap placed on the tube, according to one embodiment of the present invention.

FIG. 11 is a perspective view of a relatively non-rigid collection container, according to one embodiment of the present invention.

FIG. 11A is a perspective view of a relatively non-rigid collection container, according to an alternative embodiment of the present invention.

FIG. 12 is a front view of a handle of a relatively non-rigid collection container, according to one embodiment of the present invention.

FIG. 13 is a top view of a handle of a relatively non-rigid collection container, according to one embodiment of the present invention.

FIG. 13A is a photograph depicting a perspective view of a relatively non-rigid collection container, according to one embodiment of the present invention.

FIG. 14 is a front view of a sterilized of a point-of-use collection tray package, according to one embodiment of the present invention.

FIG. 15 is a side view of a sterilized of a point-of-use collection tray package, according to one embodiment of the present invention.

FIG. 15A is a photograph depicting a perspective view of a point-of-use collection tray package, according to one embodiment of the present invention.

FIG. 15B is a photograph depicting a perspective view of a point-of-use collection tray package with the collection tray removed from the packaging, according to one embodiment of the present invention.

FIG. 16 is a photograph depicting a top view of a relatively non-rigid collection container containing medical instruments, according to one embodiment of the present invention.

FIG. 17 is a flow diagram depicting a method of using a collection tray, according to one embodiment of the present invention.

FIG. 18 is a flow diagram depicting a method of collecting medical instruments, according to an alternative embodiment of the present invention.

### DETAILED DESCRIPTION

The system and method of the present invention relates to containers for retention, transport, and in some embodiment, cleansing of used medical devices. The system includes two types of containers: a relatively rigid container for smaller used medical devices and used sharps and a relatively flexible container for larger used medical devices.

Generally, the containers of the present invention allow personnel at a point of use, such as an operating room or the like, to open a sterilized package and collect used medical devices, including single-use devices, at the point of use, rather than discarding the used devices. Once a procedure or operation or the like is complete, the collected devices are ready for the next step in processing, without the need to sort through an amount of material that could contain hazardous or other unsafe or unsanitary content. Since the collection process is greatly simplified, the yield for collection of single-use medical devices should increase, and more devices will be reprocessed, resulting in economic advantage.

FIG. 1 is a perspective view of a relatively rigid collection container 100, according to one embodiment of the present invention. The container 100 according to one embodiment is a relatively rigid tube 100 configured to receive, hold, and transport used medical devices. Further, the container 100 is also configured to allow for washing the devices. Alternatively, the relatively rigid container 100 can have any known shape or configuration for holding and transporting smaller used medical devices and used sharps while substantially retaining its shape. For purposes of the present application, the term “tube” is intended to include any relatively rigid container of any shape. Further, “relatively rigid” is intended to mean having the characteristic of substantially retaining shape. Thus, a relatively rigid container is any container that substantially retains its shape and is not foldable.

The container 100 depicted in FIG. 1 has a substantially cylindrical body 102 consisting of plastic mesh and two removable end caps 104. The body 102, alternatively, is made up of any relatively rigid, porous material having openings large enough to allow a substantially free flow of fluid therethrough but small enough so that the devices which are collected and transported in the tube 100 do not extend beyond the mesh. Further, the body comprises a material with a composition appropriate in strength and structure to contain sharp objects. The end caps 104 fit snugly on the ends 106 of the body 102 so that the ends do not slip off of the body 102 without the application of some force.

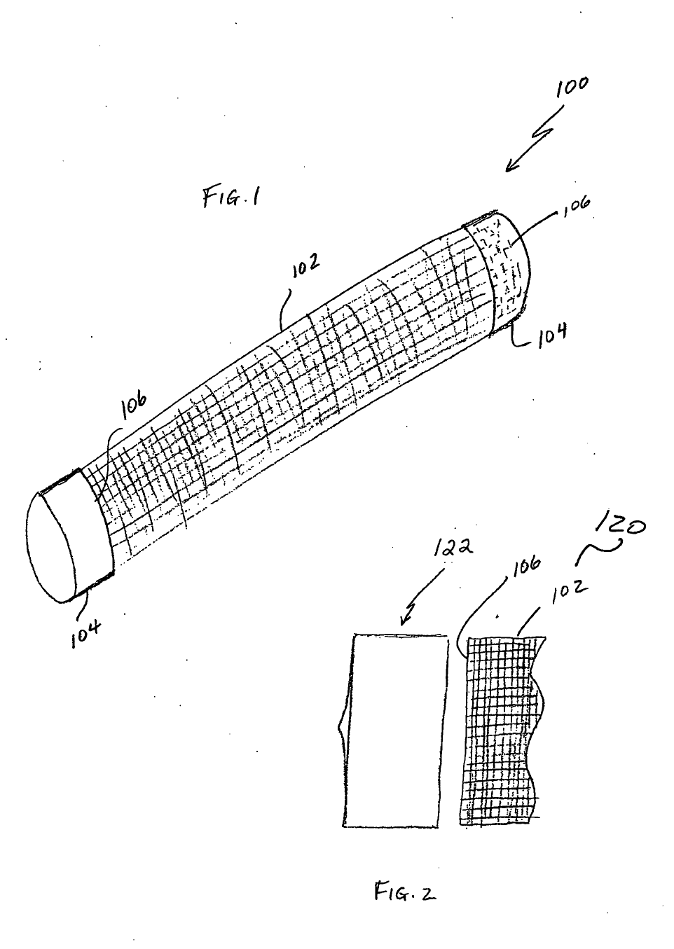

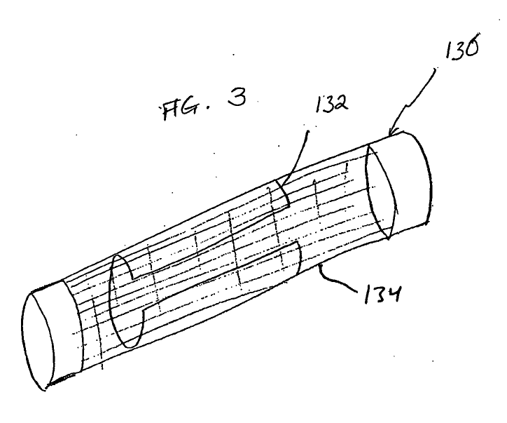

FIG. 2 is a side elevation view of a portion of a relatively rigid collection container 120, according to one embodiment of the present invention. The end cap 123 is configured in FIG. 2 to fit snugly over tube 102 to cover the tube end 106 and thus provide a secure attachment between the end cap 122 and the tube 102 that cannot be removed without some application of force. Alternatively, the end cap 122 is removably attached to the tube 102 via threads or any other known attachment mechanism. For purposes of this application, “cap” or “end cap” can be any known lid, cover, or any other type of closure that can be used in conjunction with the collection container of the present invention.

According to an alternative aspect of the invention, the tube 102 has an opening at one end requiring an end cap, but has no opening at the other end of the tube 102. The closed end of the tube 102 can be mesh or, alternatively, it can be solid.

In a further alternative, the tube body is made of a solid material and is not porous, and the end caps are made of a porous material to allow cleansing of the used medical devices contained within the tube. In yet another alternative, if the medical devices are cleansed or decontaminated prior to placement in the container, the container can be solid with solid end caps.

According to one embodiment, the tube 102 is sized to accommodate the types of single use medical devices intended to be collected. Alternatively, the tube 102 can be varied in size to accommodate different sizes of devices to be collected. In a further alternative, the tube 102 is sized so as to be easily held in a single human hand, thus allowing the user to use the other hand to load the tube.

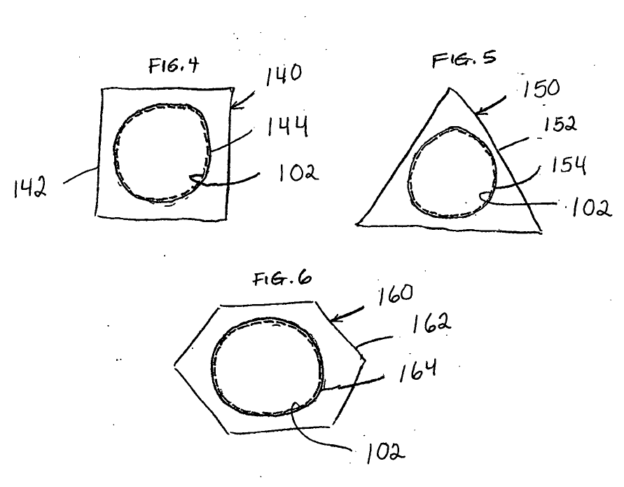

FIG. 3 depicts a collection tube 130, according to an alternative embodiment of the present invention. The tube has a plastic liner 132 positioned in the tube body 134. The liner is a flexible sheet of material made of plastic or any known material suitable as a liner. The liner 132 extends partially around the inner circumference of the tube 134. Alternatively, the liner 132 extends entirely around the inner circumference of the tube 134. The liner 132 can, according to one embodiment, serve as additional protection against a used medical device within the tube projecting out of the tube body 134, especially a sharp device that may scratch, cut, stab, prick, or otherwise injure a user.

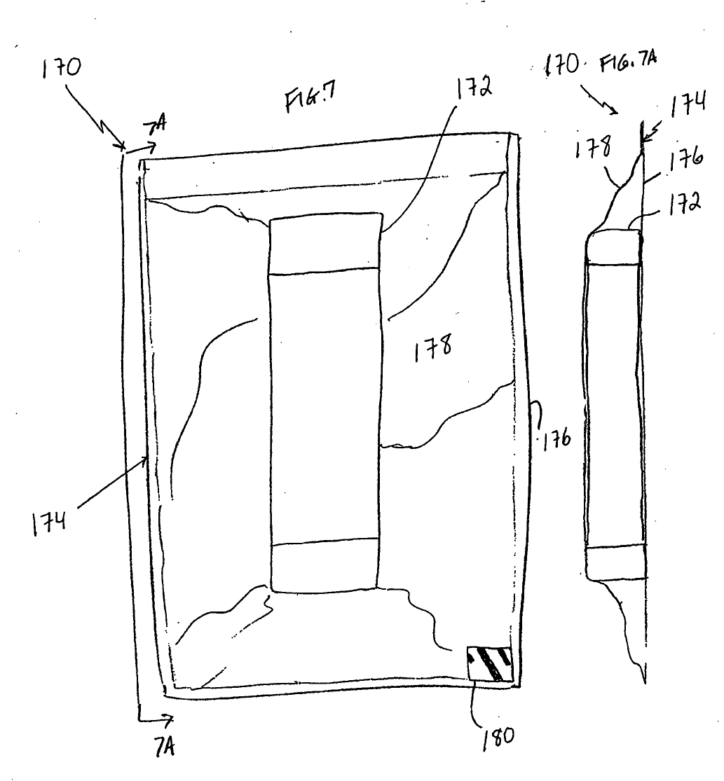

FIGS. 4, 5, and 6 depict end elevations of end caps 140, 150, and 160, respectively, according to alternative embodiments of the present invention. The end caps 140, 150, and 160 are shaped so as to substantially prevent rolling of a capped tube if the tube is set on a flat or partially inclined surface.

FIG. 4 depicts a substantially square or parallelogram-shaped end cap 140 denoted by the outline 142. The cap 140 has an interior sleeve 144 configured to receive a tube such as tube 102 such that the cap 140 is secured to the tube 102. When the tube 102 with cap 140 is set down on a flat surface, or even a partially inclined surface, the capped tube will not roll off of the surface due to the external outline 142 of the cap 140.

FIG. 5 depicts a substantially triangular-shaped end cap 150 denoted by the outline 152. The cap 150 has an interior sleeve 154 configured to receive a tube such as tube 102 such that the cap 150 is secured to the tube 102. When the tube 102 with cap 150 is set down on a flat surface, or even a partially inclined surface, the capped tube will not roll off of the surface due to the external outline 152 of the cap 150.

FIG. 6 depicts a substantially hexagonal-shaped end cap 160 denoted by the outline 162. The cap 160 has an interior sleeve 164 configured to receive a tube such as tube 102 such that the cap 160 is secured to the tube 102. When the tube 102 with cap 160 is set down on a flat surface, or even a partially inclined surface, the capped tube will not roll off of the surface due to the external outline 162 of the cap 160.

Alternatively, the end cap or end caps can have any known shape that would prevent rolling of the capped tube. Other exemplary, non-limiting end cap shapes include ovoid, rectangular, rhomboid, octagonal, and the like.

In accordance with one aspect of the present invention, a relatively rigid collection container of the present invention is packaged in a sterile package to be made available in an emergency room or operating room or any other point of use. FIGS. 7 and 7A depict a front view and a side view, respectively, of a point-of-use collection tube package 174, according to one embodiment of the present invention. The package 174 contains a tube 172. The package 174 according to one embodiment has an opaque back sheet 176 and a transparent front sheet 178 surrounding the tube 172. Alternatively, the back sheet 176 and front sheet 178 are made of any known packaging material. The package 174 is configured to be capable of maintaining a sterilized environment for the tube 172 until the package 174 is opened. According to one embodiment, the interior of the package 174 and the tube 172 are sterilized by an ethylene oxide sterilization process. Alternatively, the tube 172 and interior of the package 174 are sterilized by any known sterilization method.

According to one embodiment, a sterilization indicator 180 is provided with the package 170. The indicator 180 is configured to indicate whether the package 170 is sterile. In one aspect of the invention, the indicator 180 is a lines tape that exhibits one color when it is sterile and another color when it is not sterile. After sterilization, the package 170 and the tube 172 remain sterile until the package 170 is opened. When the package 170 has been sterilized, it can be placed at the point of collection for single-use medical devices, such as in an operating room or the like. Then, when the single-use medical devices are used, they can be immediately placed into a collection container such as the collection tube 172 by simply opening the package 170, removing the tube 172, and placing the device or devices into the tube 172.

In one aspect of the present invention, certain embodiments of the relatively rigid collection container 184 are configured to allow for decontamination or cleansing of the used medical devices without removing them from the container. FIG. 8 depicts a bath 186, according to one embodiment of the present invention. The bath 186 contains a decontaminating or cleansing solution 188. Alternatively, the bath 186 contains any liquid or solution that cleanses or decontaminates used medical devices. The bath 186 is configured to hold one or more tubes 184. The porous nature of the tube body 185, as discussed above, allows the solution to pass into the tube 184 and contact the devices collected inside the tube 184.

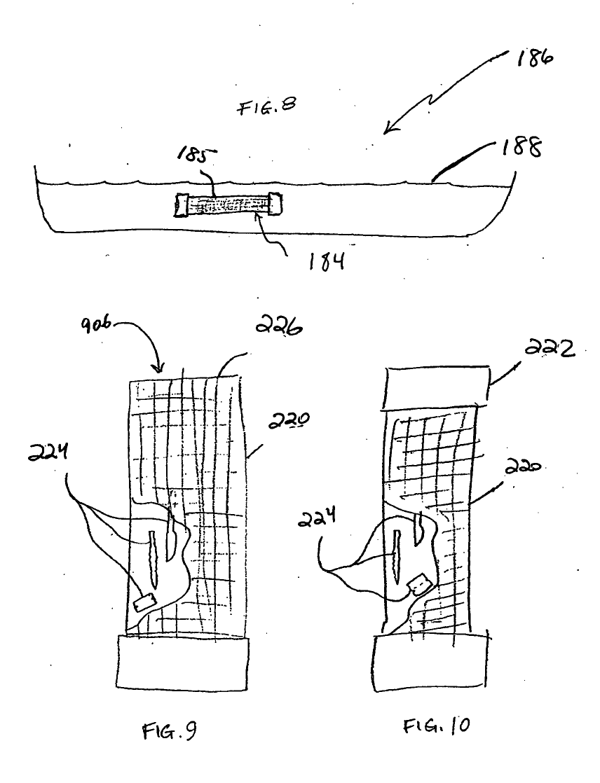

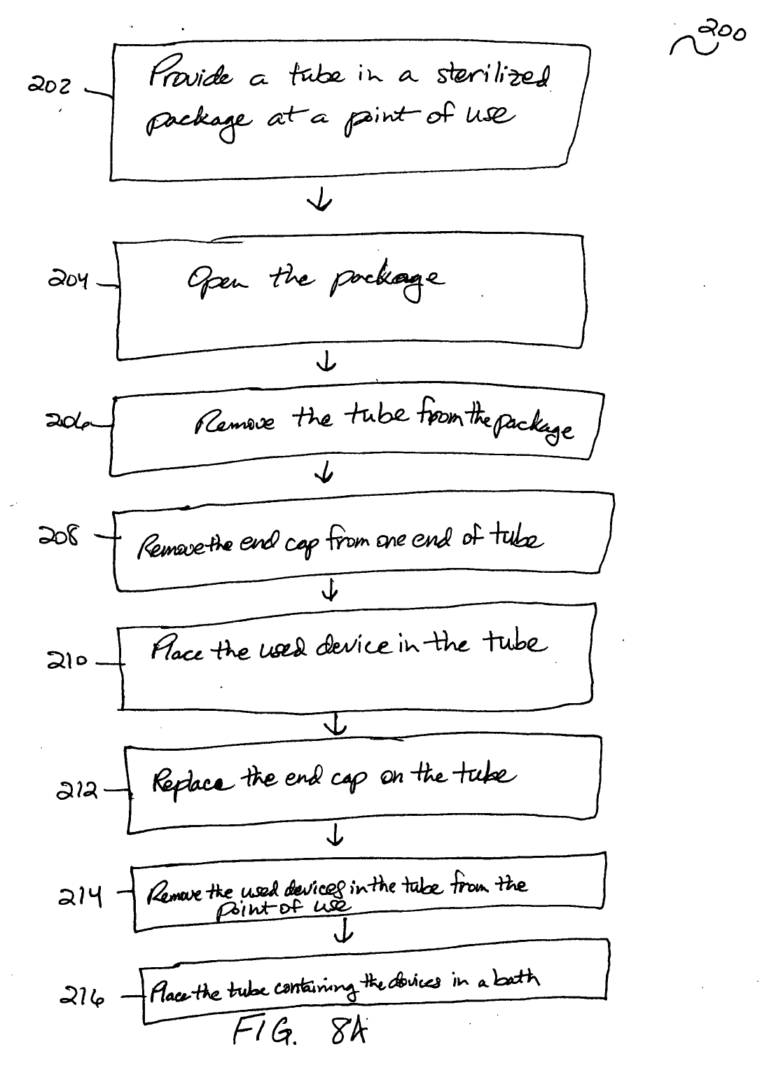

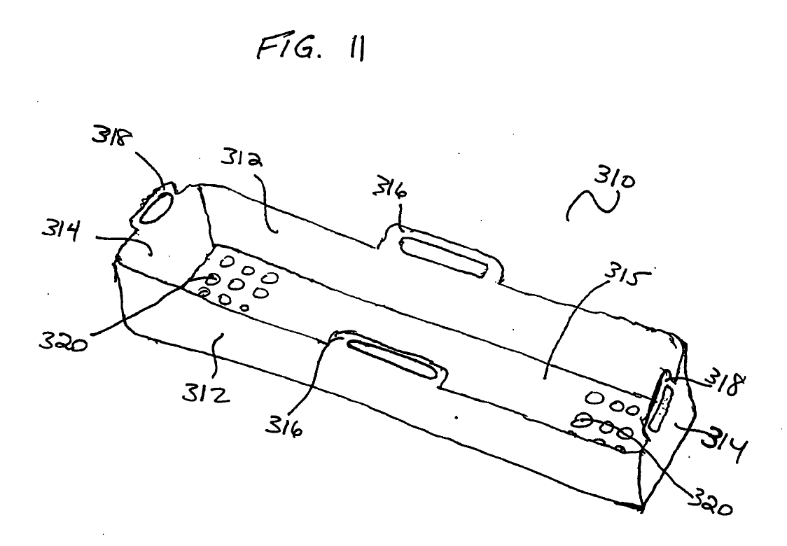

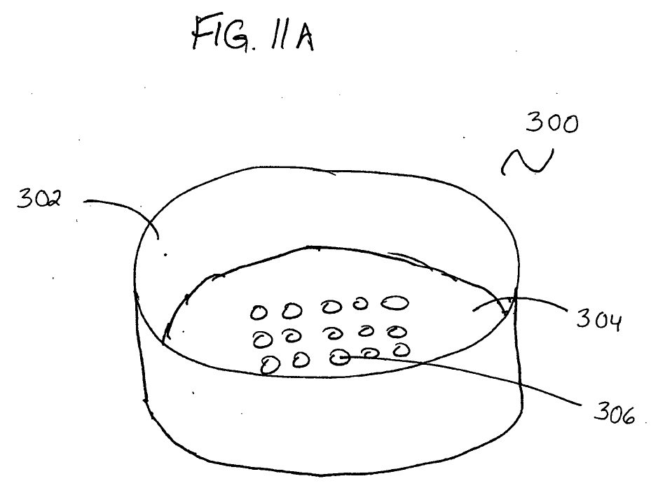

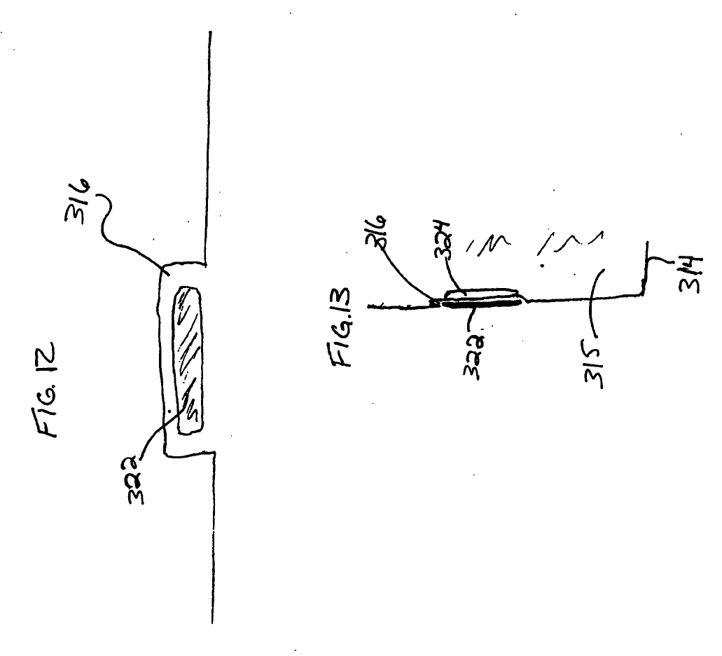

FIG. 8A depicts a method of using a relatively rigid container 40, according to one embodiment of the present invention. In use, a sterilized package containing a tube 220, as shown in FIGS. 9 and 10, is provided at a point of use for the devices that are to be collected for reprocessing (block 202). Such a point of use may be an operating room, an ambulance, or the like. When a device to be reprocessed is used and is ready for collection, a package is opened (block 204), the tube 220 removed (block 206), an end cap 222 removed from one end 226 of the tube 220 (block 208), the device or devices 224 are placed in the tube 220 as depicted in FIG. 9 through the open end 226 of the tube 220 (block 210), the end cap 222 is replaced on the tube 220 (block 212) as further shown in FIG. 10, and the tube 220 containing the devices 224 is removed from the point of use (block 214).

Upon removal from the point of use, the devices are often subsequently washed or decontaminated. In accordance with one aspect of the invention, the devices can be washed or decontaminated while remaining in a relatively rigid container of the present invention. That is, the embodiments of the present invention allow for a decontamination process without removing the single use medical devices from the tube. According to one alternative embodiment, therefore, a tube containing a collected device is placed in a bath (block 216) and thus the used medical devices are washed without removing them from the tube. The decontamination or cleansing steps may take place before or after removing the devices from the point of use.

FIG. 11 is perspective view of a relatively non-rigid collection container 310 according to one embodiment of the present invention. The container 310 according to one embodiment is configured to receive, hold, and transport used medical devices. Further, the container 310 is also configured to allow for washing the devices. The container 310 has two side walls 312 and two end walls 314 attached to a base 315. There are several holes 320 in the base. Handles 316, 318 are formed at the top of each of the side walls 312 and end walls 314. For purposes of the present application, the term “tray” is intended to include any relatively non-rigid container of any shape. Further, “relatively non-rigid” is intended to mean having the characteristic of being substantially flexible, foldable, pliable, or malleable. Thus, a relatively non-rigid container is any container that is generally pliable, flexible, malleable, or foldable.

FIG. 11A is perspective view of a collection tray 300 according to an alternative embodiment of the present invention. The tray 300 is round and is configured to receive, hold, transport and wash used medical devices. The tray 300 has a wall 302 attached to a base 304. There are several holes 306 in the base. According to one embodiment, the tray 300 has handles (not shown). Alternatively, the collection tray 300 has an oval shape, a rectangular shape, or any other known shape.

The base 315 and walls 312, 314, in accordance with one aspect of the invention, consist of a flexible or pliable material that allows the container 310 to be folded into smaller dimensions. According to one embodiment, the material is a plastic blend made up of polyethylene and polypropylene. Alternatively, the material is any known flexible material that can be used in a container. In yet another alternative, the base 315 and the walls 312, 314 consist of a non-flexible material.

The walls 312, 314 according to one embodiment are foldable or enclosable such that the walls 312, 314 overlap with each other, thus covering any devices contained within the container 310. According to a further embodiment, the container 310 has a fastening component (not shown) configured to fasten the overlapping walls in a “closed” position. The fastening component can be any known mechanism for fastening a portion of a container so as to fasten an opening closed.

In accordance with one embodiment, the container 310 is sized to receive, hold, and transport used medical devices. That is, the container 310 can be of any dimensions required in order to receive and transport used medical devices of any desired size. According to one aspect of the invention, the container 310 is sized to receive, hold, and transport any used medical devices that cannot fit into the collection container disclosed and described below.

According to one embodiment, the base 315 has about 60 holes 320, each having a diameter of about ⅜ of an inch. Alternatively, the base 315 has from about 1 hole 320 to about 200 holes 320. The diameter of the holes 320 can range from about 1/16 of an inch to about 1.5 inches. Alternatively, the base 315 has any number of apertures 320 of any size such that they allow for a substantially free flow of fluid therethrough without allowing any devices placed in the container 310 to escape from the container 310 through the apertures 320.

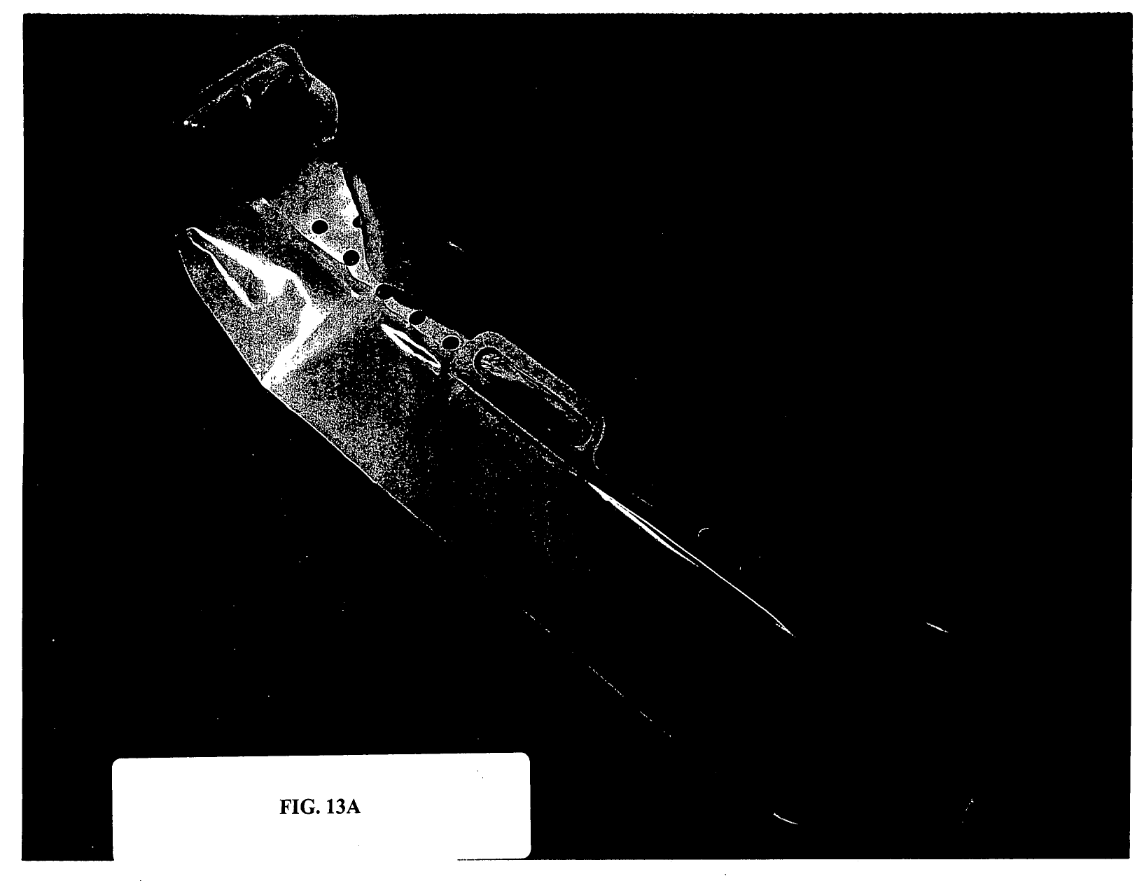

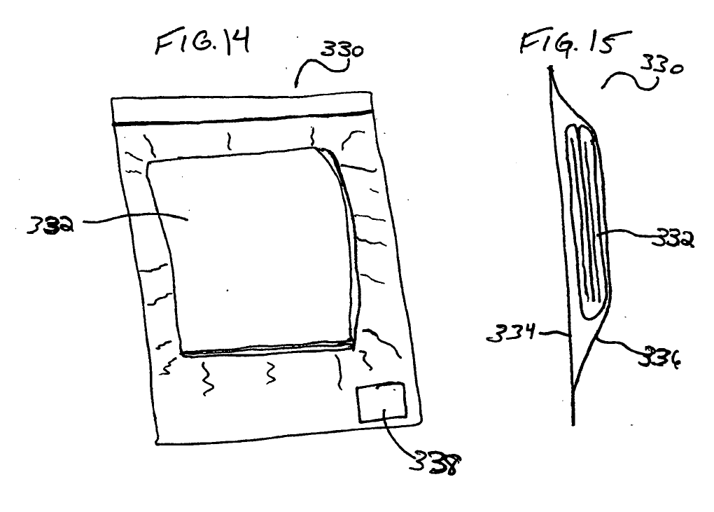

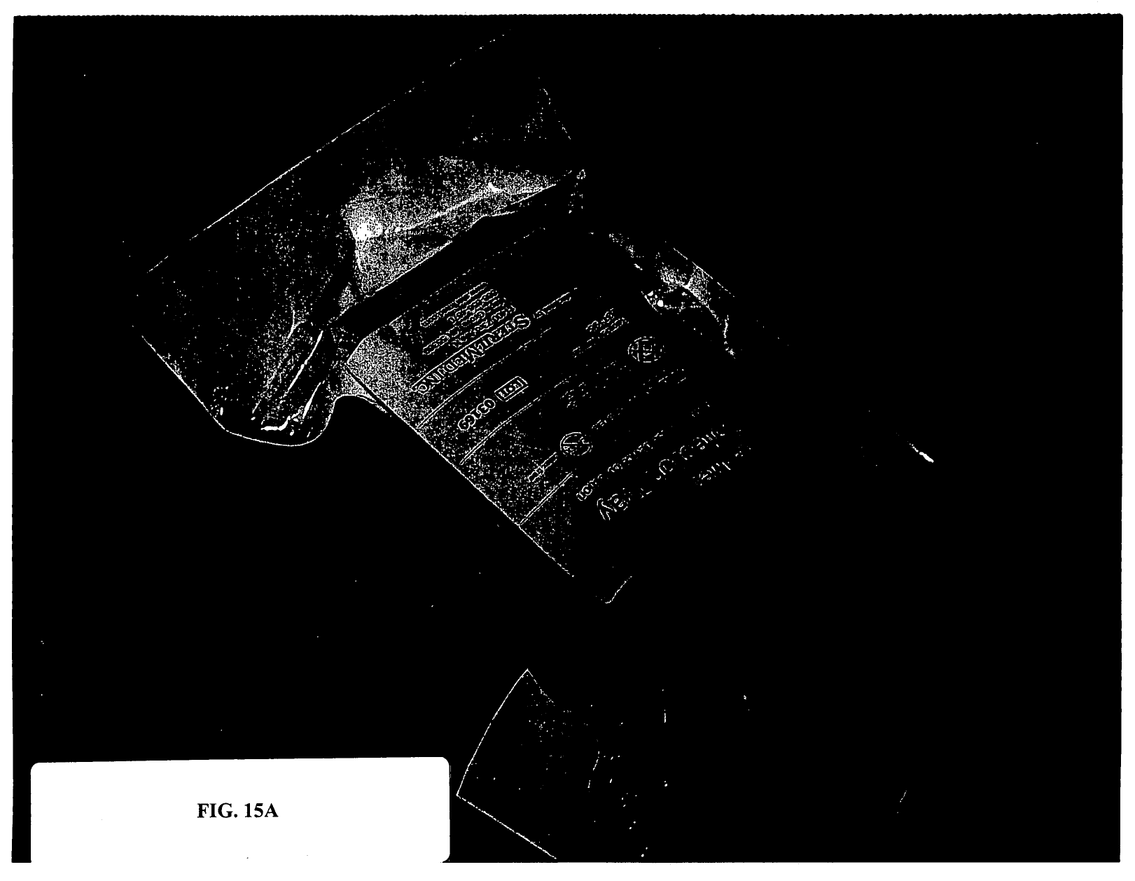

FIGS. 12 and 13 depict a front and top view, respectively, of a handle 316 of a tray 310, according to one embodiment of the present invention. The following description can also apply to the handles 318 formed in the top of each end wall 314. The handle 316 is formed with an indentation 322 in the handle 316 designed to allow for ease of grasping the handle 316. The indentation 322 in the handle 316 results in a bulge 324 on the opposite side of the handle 316. In accordance with one embodiment, the indentation 322 is on the exterior portion of the handle 316. Alternatively, the indentation 322 can be on the interior portion of the handle 316. In a further alternative, the handles 316, 318 have holes (not shown) instead of indentations 322. In yet another alternative, the handles 316, 318 have any known configuration allowing for easier transport of the container 310. Alternatively, the container 310 does not have handles.

FIG. 13A is a color photograph depicting a collection tray, according to one embodiment of the present invention.

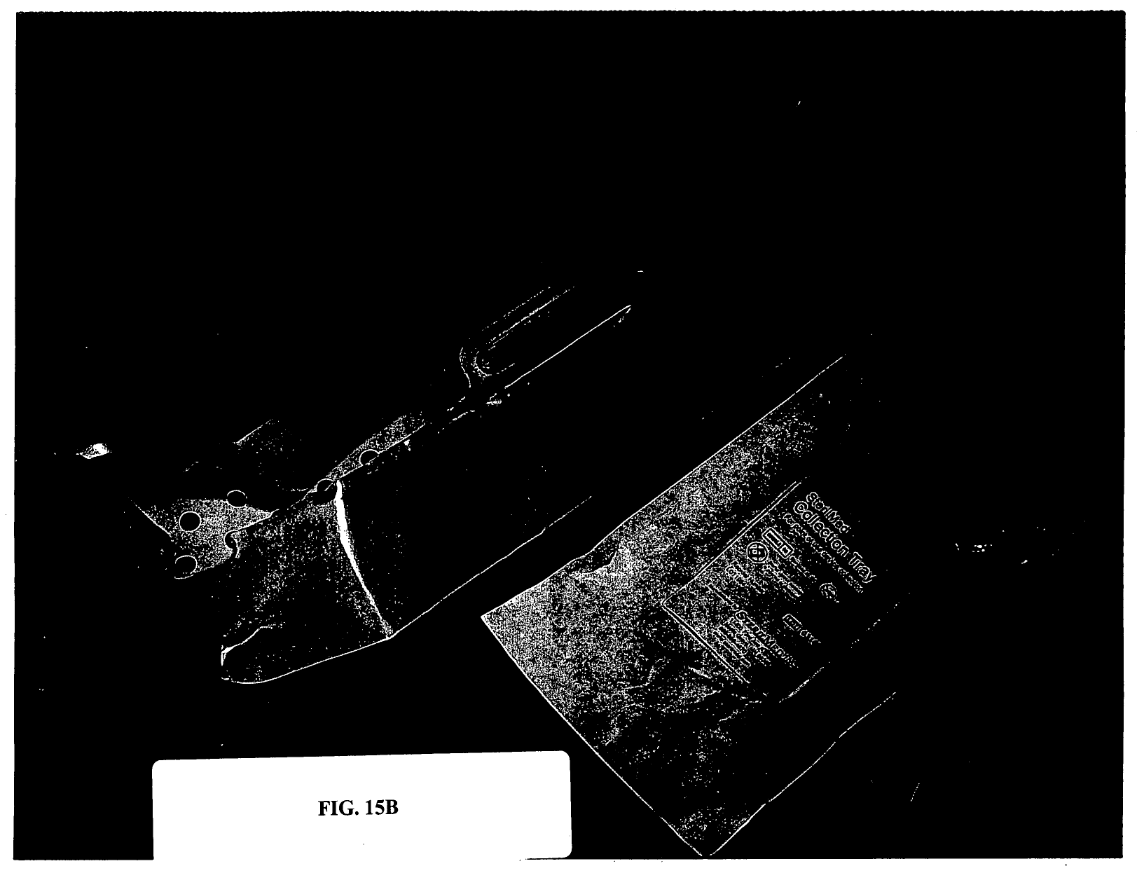

In accordance with one aspect of the present invention, a container of the present invention is folded into smaller dimensions and packaged in a sterile package to be made available in an emergency room or operating room or any other point of use. FIGS. 14 and 15 depict a front view and a side view, respectively, of a point-of-use collection tray package 330, according to one embodiment of the present invention. The package 330 contains a folded tray 332. The package 330 according to one embodiment comprises an opaque back sheet 334 and a transparent front sheet 336 surrounding the folded container 332. Alternatively, the back sheet 334 and front sheet 336 are made of any known packaging material. The package 330 is configured to be capable of maintaining a sterilized environment for the container 332 until the package 330 is opened. According to one embodiment, the interior of the package 330 and the container 332 are sterilized by an ethylene oxide sterilization process. Alternatively, the container 332 and interior of the package are sterilized by any known sterilization method. FIG. 15A is a photograph depicting a perspective view of a point-of-use collection tray package, according to one embodiment of the present invention.

According to one embodiment, a sterilization indicator 338 is provided with the package 330. The indicator 338 is configured to indicate whether the package is sterile. In one aspect of the invention, the indicator 338 is a lines tape that exhibits one color when it is sterile and another color when it is not sterile. After sterilization, the package 330 and the tray 332 remain sterile until the package 330 is opened. When the package 330 has been sterilized, it can be placed at the point of collection for single-use medical devices, such as in an operating room or the like. Then, when the single-use medical devices are used, they can be immediately placed into a collection container such as the collection tray 332 by simply opening the package 330, removing the tray 332, and placing the device or devices into the tray 332.

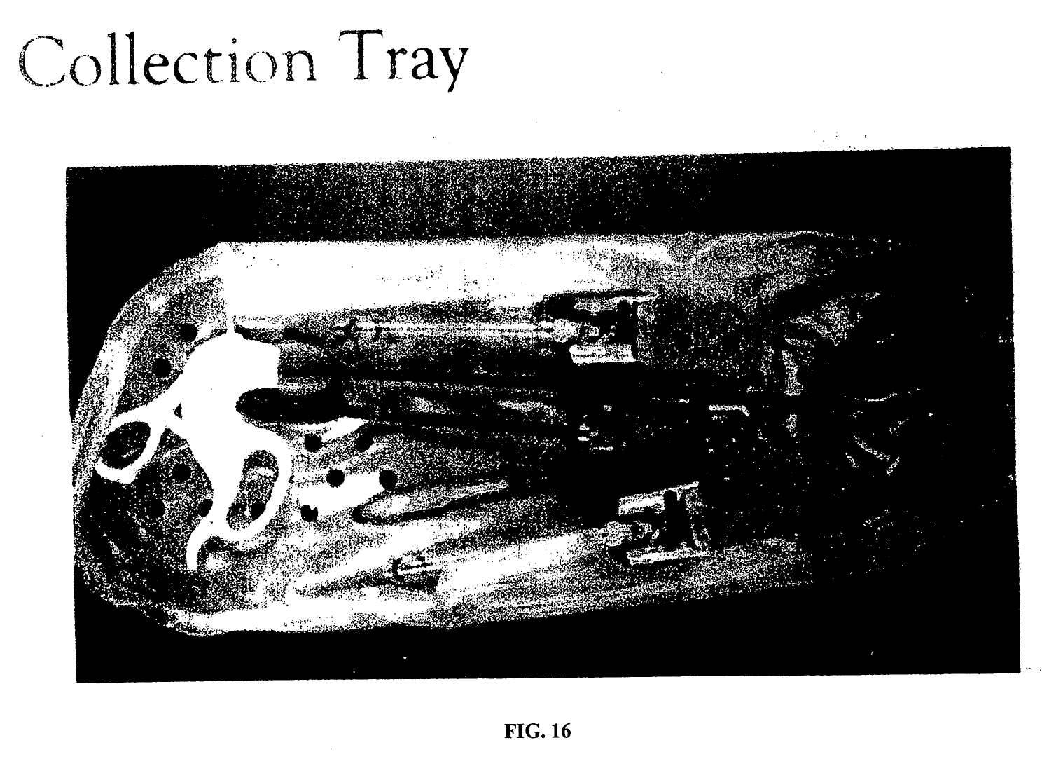

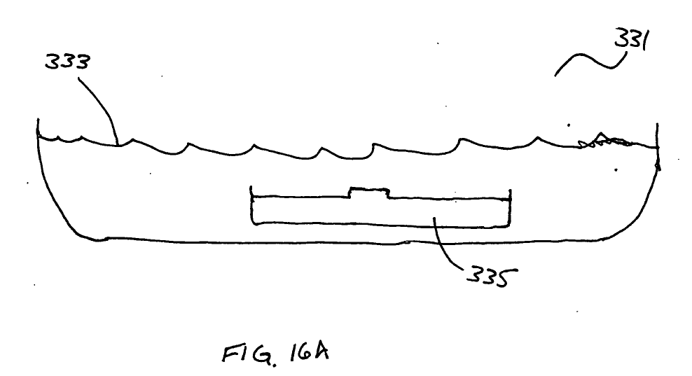

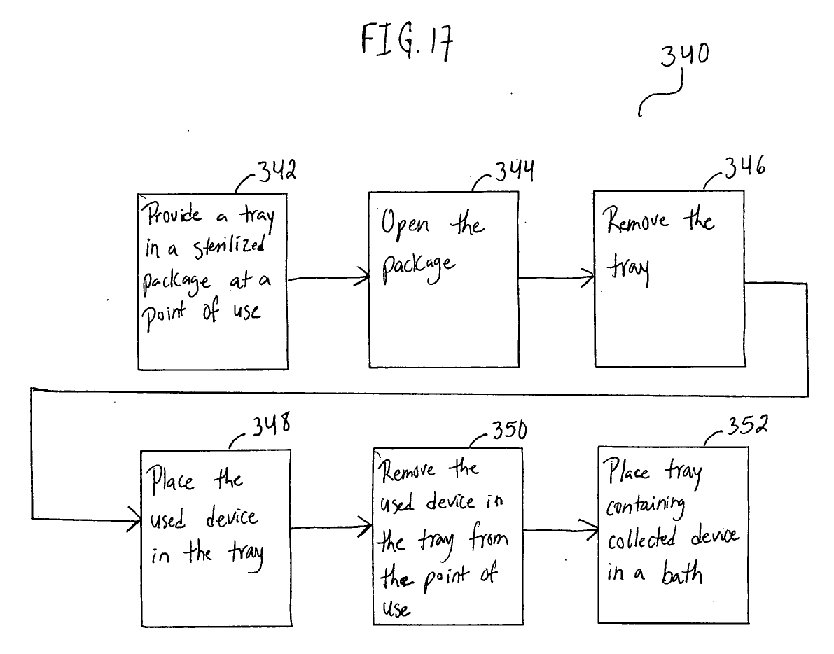

In one aspect of the present invention, certain embodiments of the relatively non-rigid collection container are configured to allow for decontamination or cleansing of the used medical devices without removing them from the container. FIG. 16A depicts a bath 331, according to one embodiment of the present invention. The bath 331 contains a decontaminating or cleansing solution 333. Alternatively, the bath 331 contains any liquid or solution that cleanses or decontaminates used medical devices. The bath is configured to hold one or more trays 335. The opening at the top of the tray 335 and the holes in the base of the tray 335 allows the solution to pass into the tray 335 and contact the devices collected inside the tray 335.

FIG. 17 depicts a method of using a relatively non-rigid container 340, according to one embodiment of the present invention. In use, a sterilized package containing a folded tray is provided at a point of use for the devices that are to be collected for reprocessing (block 342). Such a point of use may be an operating room, an ambulance, or the like. When a device to be reprocessed is used and is ready for collection, a package is opened (block 344), the tray removed as shown in FIG. 15B (block 346), the device or devices are placed in the tray as depicted in FIG. 16 (block 348), and the tray containing the devices is removed from the point of use (block 350).

Upon removal from the point of use, the devices are often subsequently washed or decontaminated. In accordance with one aspect of the invention, the devices can be washed or decontaminated while remaining in a relatively non-rigid container of the present invention. That is, the embodiments of the present invention allow for a decontamination process without removing the used medical devices from the tray. According to one embodiment, therefore, a tray containing a collected device or devices is placed in a bath (block 352) and thus the used medical devices are washed without removing them from the tray. The decontamination or cleansing steps may take place before or after removing the devices from the point of use.

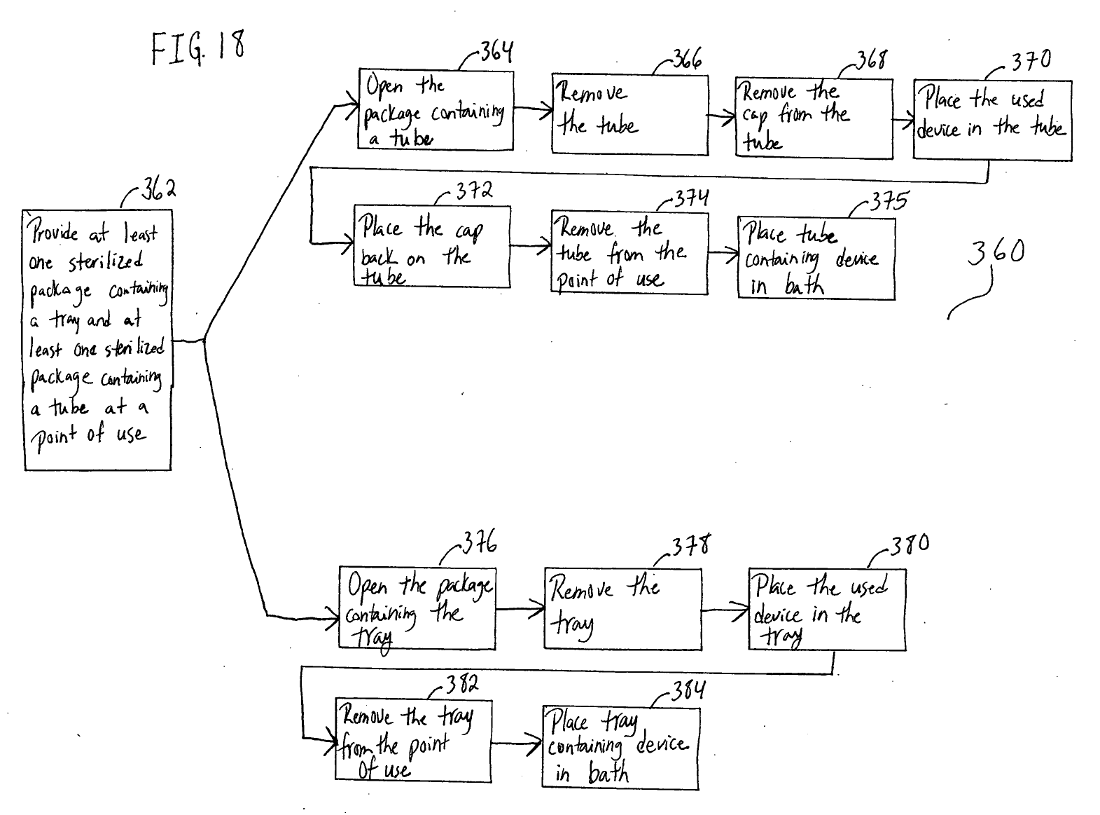

FIG. 18 depicts an alternative method of collecting used medical devices 360, according to one embodiment of the present invention. At least one sterilized package containing a folded tray and at least one sterilized package containing a collection tube are provided at a point of use for the devices that are to be collected for reprocessing (block 362). When a device or devices that fit in a collection tube are used and are ready for collection, a package is opened (block 364), the tube is removed (block 366), the cap is removed from the tube (block 368), the device or devices are placed in the tube (block 370), the cap is placed back on the tube (block 372) and the tube containing the devices is removed from the point of use (block 374). Alternatively, the tube is re-capped and retained for further collection procedures. In a further alternative embodiment, the devices are placed in a decontamination bath while still in the tube (block 375). The bath step can take place either before or after removal from the point of use.

When a device or devices that do not fit in a collection tube are used and are ready for collection, a tray package is opened (block 376), the tray removed (block 378), the device or devices are placed in the tray (block 380), and the tray containing the devices is removed from the point of use (block 382). Alternatively, the tray is retained for further collection procedures. In a further alternative embodiment, the devices are placed in a decontamination bath while still in the tray (block 384). The bath step can take place either before or after removal from the point of use.

Although the present invention has been described with reference to preferred embodiments, persons skilled in the art will recognize that changes may be made in form and detail without departing from the spirit and scope of the invention.

What is claimed is:

1. A collection container for used medical devices comprising:
(a) a flexible base defining at least one aperture; and
(b) at least one flexible wall coupled to the base, the at least one wall defining an opening in the container.

2. The collection container of claim 1, wherein the at least one aperture is configured to allow passage of fluid.

3. The collection container of claim 1, wherein the container is foldable.

4. The collection container of claim 1 further comprising at least one handle coupled to the container.

5. The collection container of claim 1 wherein the base and the at least one wall comprise a flexible plastic comprising polyethylene and polypropylene.

6. The collection container of claim 1 further comprising a sterile packaging configured to receive the collection container.

7. A collection package for the collection of used medical devices, comprising:
(a) a collection container comprising:
(i) a flexible base defining at least one aperture; and
(ii) at least one flexible wall coupled to the base, the at least one wall defining an opening in the container; and
(b) a package configured to removably receive the collection container, the packager further configured to maintain sterility of the collection container before removal.

8. The collection package of claim 7, wherein the at least one aperture is configured to allow passage of fluid.

9. The collection container of claim 7, wherein the container is foldable.

10. The collection container of claim 9, wherein the package is configured to receive the collection container in a folded configuration.

11. The collection container of claim 7 further comprising at least one handle coupled to the container.

12. The collection container of claim 7 wherein the base and the at least one wall comprise a flexible plastic comprising polyethylene and polypropylene.
